(19) United States
(12) Patent Application Publication
(10) Pub. No.: US 2007/0265210 A1
(43) Pub. Date: Nov. 15, 2007

(54) Administration of C-glycoside compounds for protecting and/or activating gammadeltaT lymphocytes

(75) Inventor: Lionel Breton, Versailles (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/783,410

(22) Filed: Apr. 9, 2007

(30) Foreign Application Priority Data
Apr. 7, 2006 (FR) 0651269

(51) Int. Cl. A01N 43/04; A61P 17/14; A61K 31/70

(52) U.S. Cl. 514/25

(57) ABSTRACT

C-glycoside compounds having the general formula (I): 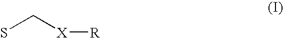 are useful for protecting and/or stimulating the activity and/or the proliferation of gamma-delta T lymphocyte cells (γδT cells), and are advantageously formulated into cosmetic/pharmaceutical compositions suited for promoting skin tissue repair, for re-equilibrating epidermal proliferation and differentiation disorders which appear with lack of sleep, and for improving the appearance of the head of hair and limiting hair loss.

### CROSS-REFERENCE TO PRIORITY APPLICATION

This application claims priority under 35 U.S.C. § 119 of FR 06/51269, filed Apr. 7, 2006, hereby expressly incorporated by reference and assigned to the assignee hereof.

### CROSS-REFERENCE TO COMPANION APPLICATION

Copending U.S. Patent Application No. [Attorney Docket No. 1016800-000848], filed concurrently herewith, also expressly incorporate by reference and also assigned to the assignee hereof.

### BACKGROUND OF THE INVENTION

1. Technical Field of the Invention

The present invention relates to the administration of at least one compound of general formula (I): 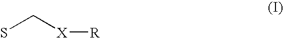
as an agent for protecting and/or stimulating the activity and/or the proliferation of gamma-delta T lymphocyte cells (γδT cells), formulated into compositions containing a cosmetically or pharmaceutically acceptable medium.

The subject compositions are useful for promoting skin tissue repair, for re-equilibrating epidermal proliferation and differentiation disorders which appear with lack of sleep, and for improving the appearance of the head of hair and limiting hair loss.

2. Description of Background and/or Related and/or Prior Art

The human skin consists of a superficial compartment, the epidermis, and a deep compartment, the dermis. The epidermis is mainly composed of three cell types, which are keratinocytes (predominant), melanocytes and Langerhans cells. Each of these cell types contributes by virtue of its own functions to the essential role played in the organism by the skin, in particular the role of protecting the organism against external attacks. The dermis provides a solid support for the epidermis. It is also its feeder element. It consists mainly of fibroblasts and of an extracellular matrix which is itself composed mainly of collagen, elastin and a ground substance. Leukocytes, mast cells and tissue macrophages are also found therein. Finally, blood vessels and nerve fibers pass through the dermis.

The skin constitutes a barrier against external attacks, in particular chemical and mechanical challenges, and, in this respect, a certain number of defense reactions against the environment (climate, ultraviolet rays, tobacco, pollutants, etc.) and/or xenobiotics (such as, for example, certain medicaments) take place within it.

In individuals who are in good health, cells damaged by external attacks are eliminated by the cutaneous immune system. However, for example, when an individual experiences a lack of sleep, the immune system is immunodepressed and cannot play its role in monitoring the skin.

Human lymphocytes are characterized by a specific antigen receptor (TCR, T cell receptor). 95% of the lymphocyte population consists of lymphocytes in which the T lymphocyte antigen receptors are transmembrane heterodimers composed of an alpha and beta chain (αβT). Only approximately 5% of lymphocytes express gamma and delta chains (γδT); this population was demonstrated by Tonegawa et al., in 1984.

γδT lymphocytes are mainly located in the blood (for the Vδ2 subtype) and in the epithelia: epidermis and mucous membranes (for the Vδ1 resident subtype). They express CD3 receptors and gamma-delta TCR receptors (respectively with two variable regions depending on whether they are circulating: Vγ9Vδ2, or resident: Vγ9Vδ1) and are often CD4(−)-CD8(−), their activation is not major histocompatibility complex (MHC)-restricted and a role in the regulation of cell homeostasis is attributed to them. The gamma chain is encoded by chromosome 7 and the delta chain by 14.

γδT lymphocytes constitute a T lymphocyte population for which the analysis of the structural characteristics has modified the knowledge of interactions from the T cell and its antigen. The importance of the immunoregulatory functions exercised by these cells opens up new perspectives in understanding the mechanisms which contribute to maintaining the homeostasis of the immune system. γδT lymphocytes appear to be involved very early in the regulation of innate immunity.

Recent studies demonstrate that specific T cells play an important role in epidermal homeostasis (Nature Immunology, 6,1 2005, 73-76). In fact, the γδT lymphocytes of the skin and/or the mucous membranes induce the secretion and release of IGF1 in the skin, and this release of IGF1 results in a control of epidermal homeostasis, in particular the balance from epidermal cell proliferation and differentiation.

The skin undergoes constant remodeling and certain of these transformations observed over time are in particular the result of a modification of the natural secretion, by the organism, of hormones (growth hormones, prolactin, oestrogen, etc.) and of growth factors (TGFα and TGFβ, EGF, IGF, VEGF, etc.).

The role of these hormones and/or of these growth factors is all the more important since, over time, their release and their impact on the target tissues decreases, and tissue growth stabilizes, while the increase in matrix degradation is no longer compensated for by the regenerating action of these hormonal and/or growth factors.

Among these growth factors, IGFs (insulin growth factors) play a predominant role. These factors were discovered while the mechanism of action of growth hormone (GH), which stimulates the growth of all tissues, including skin tissues, was being explored. Growth hormone (GH) consists of 191 amino acids linked in a specific sequence and is secreted by the anterior part of the pituitary gland; this secretion can be reinforced by physical exercise and by other factors. The biological role of GH is fundamental, not only for the growth of a young organism in terms of stature, but also for maintaining its integrity in adulthood. GH acts on the peripheral organs and the brain either directly, or indirectly by stimulating the synthesis of growth factors, such as insulin-like growth factors (IGF I and II) or epidermal growth factor (EGF), or that of their receptors. The direct action of GH is of anti-insulin type by promoting lipolysis in adipose tissues.

By means of IGF1, GH stimulates the incorporation of amino acids into proteins, cartilage and bone growth (statural growth), and cell proliferation in many organs, including the skin.

Growth hormone and the other somatotropic hormones, such as somatomedin C (or IGF1), are capable of maintaining the body's young appearance. They are in fact involved in controlling its metabolism, determining not only the final size of the adult body, but also giving volume, tone and firmness to the organs and tissues, particularly to the muscles. In fact, growth hormone contributes to everything which provides a good self image and a positive mental state. It firms the body and makes the back straighter, and develops the muscles of the shoulders and pelvis. It also decreases fat, especially on the stomach, and increases libido and sexual energy, hair regrowth and color, and skin elasticity. Perhaps less apparent, but just as beneficially, its effects can be seen in a greater ability to withstand exercise, sleep which is less prolonged and more profitable, an equilibrated arterial blood pressure, and better visual, auditory and cerebral acuity.

In all the mammals studied, including humans, GH is secreted in a pulsatile manner and this characteristic constitutes a determining factor for a large number of biological effects of the hormone.

The causes of the age-related decline in GH are poorly understood. In humans, from puberty, a decrease in GH secretion of the order of 10% every ten years is observed. During aging, loss of muscle mass, accumulation of adipose tissue, bone demineralization and the loss of tissue regeneration capacity are concomitant with the decrease in GH secretion. The latter promotes an increased catabolism-to-anabolism ratio, thus leading to a situation of imbalance which worsens the effects of aging.

Additional factors, such as weight gain, or even obesity, related to age, steroid hormone levels, a lack of sleep or a certain degree of tissue resistance to the action of GH can also be important.

In parallel, there is a notable decrease in the quality of sleep with age (decrease in slow wave sleep and in paradoxical sleep and increase in the periods and the duration of awake state which interrupt the phases of sleep). The first phenomenon of aging is a marked decrease in deep sleep (slow wave sleep or SWS), which can occur as early as the age of 36 and which is replaced by a lighter sleep. The passage from about forty years of age to an elderly age is then associated with a decrease in the amount of sleep and in the duration of the paradoxical phase (rapid eye movement or REM) and of deep sleep (non-REM). Statistics demonstrate that the population affected by sleep disorders comes to 93 million in North America, in Europe and in Japan.

It is conceivable that age-related lack of sleep or qualitative reduction in sleep contributes to hormonal modifications and to the metabolic consequences thereof In fact, pharmacological treatments which tend to increase slow wave sleep also lead to an increase in GH secretion.

Growth hormone deficiency is reflected by physical symptoms: hair loss, fine hair, thin lips and jaw bone, dehydrated skin, sagging stomach, pads of fat at the knees, etc., and psychological symptoms: constant fatigue, difficulties in controlling ones emotions, exhaustion after physical exercise, low self esteem, depression, etc.

The levels of growth hormones circulating in the blood stimulate the production, from the liver, of another hormone, IGF1 (insulin like growth factor 1), the mediator role of which allows growth hormone to develop its positive effects. It is considered to be safer to measure the level of IGF1, also called somatomedin C, than the level of GH, which is virtually undetectable during the day in humans. The liver is the most important site of IGF production, but many cells are capable of producing IGFs. Two types are conventionally described: IGF1 and IGF2. They are two peptides whose amino acid sequence is related to that of insulin, hence their name. Two receptors exist, respectively for IGF1 and for IGF2. The IGF1 receptor has a shared affinity with insulin. This is not the case of the IGF2 receptor.

With aging, the IGF level decreases and stabilizes in adulthood: this is the somatopause (D. Radman “Effects of human growth hormone in men over 60 years old”: N. Engl. J. Med., 1990, Juls; 323(1): 1-6).

It is known in the prior art that IGF1 alone stimulates keratinocyte proliferation (Neely E K—Insulin-like growth factors are mitogenic for human keratinocytes and a squamous cell carcimona—J. Invest. Dermatol., 1991 Jan; 96(1): 104-10). As regards fibroblasts, it is also known that IGF1 stimulates the synthesis of GAGs and the synthesis of collagen. Moreover, studies have demonstrated a not insignificant participation of IGFs in healing. Finally, a study carried out in vivo in humans has demonstrated that percutaneous treatment with IGF1 for one month results in an increase in skin thickness.

It has also been demonstrated that decreased IGF1 expression is associated with increased hair loss (Tang et al., 2003, J. Am. Acad. Dermatol., Aug; 49(2): 229-33).

### SUMMARY OF THE INVENTION

It has now been demonstrated that the compounds of general formula (I): 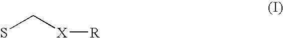
stimulate and/or protect γδT cells and that this extract can thus induce the secretion and the release of IGF1 in the skin. This control of IGF1 release contributes to maintaining epidermal homeostasis, which regulates in particular the balance from epidermal cell proliferation and differentiation.

Thus, in a first aspect, the present invention features administration of at least one compound of general formula (I): 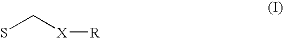
in which,

S is a monosaccharide or a polysaccharide containing up to 20 sugar units, in pyranose and/or furanose form and of L and/or D configuration, said mono- or polysaccharide having at least one hydroxyl function that is necessarily free and/or optionally one or more optionally protected amine functions;

the S—CH₂X bond is a bond of C-anomeric type;

X is a group selected from: —C—, —CH(NR₁R₂)—, —CHR′—, —C(═CHR)—;

R is a linear or branched, saturated or unsaturated alkyl, perfluoroalkyl or hydrofluoroalkyl chain, or a cycloalkyl, cycloperfluoroalkyl or cyclohydrofluoroalkyl ring member having from 1 to 18 carbon atoms, or a phenyl radical, said chain, said ring member or said phenyl radical optionally being interrupted with one or more heteroatoms selected from among oxygen, sulfur, nitrogen and silicon, and optionally substituted with at least one radical selected from —OR′₁, —SR″₁, —NR′″₁R′₂, —COOR″₂, —CONHR′″₂, —CN, halogen, perfluoroalkyl and hydrofluoroalkyl and/or at least one optionally substituted cycloalkyl, aryl or heterocyclic radical;

R′, R₁ and R₂, which may be identical or different, have the same definition as that given for R, and can also represent a hydrogen or a hydroxyl radical;

R′₂ and R′″₂, which may be identical or different, represent a hydrogen atom, or a radical selected from among a hydroxyl radical and a linear or branched, saturated or unsaturated alkyl, perfluoroalkyl and/or hydroxyfluoroalkyl radical having from 1 to 20 carbon atoms;

R′₁, R″₁, R″₂ and R′″₁, which may be identical or different, represent a hydrogen atom, or a radical selected from among a linear or branched, saturated or unsaturated alkyl, perfluoroalkyl and/or hydroxyfluoroalkyl radical having from 1 to 20 carbon atoms;
with the following provisos:

R₁ and R₂ cannot simultaneously each be a hydroxyl radical;

R′″₁ and R′₂ cannot simultaneously each be a hydroxyl radical;

when S is a D-xylose and X is ═CO, then R cannot be an unsubstituted phenyl;

when S is a D-glucose and X is the function —OH, then R cannot be a p-hydroxyphenyl; and

when S is a D-glucose and X is ═O, then R cannot be a p-methoxyphenyl or a hexyl radical;

for maintaining and/or re-establishing the balance from epidermal cell proliferation and differentiation.

### DETAILED DESCRIPTION OF BEST MODE AND SPECIFIC/PREFERRED EMBODIMENTS OF THE INVENTION

The C-glycoside compounds that are administered according to the invention, whether regime or regimen, represent a subfamily of the C-glycoside derivatives described in EP-1,345,919; they can be prepared according to the method described therein.

Preferably, the compounds of general formula (I) administered according to the invention are such that:

S is a monosaccharide or a polysaccharide having up to 5 sugar units, in pyranose and/or furanose form, and of the L and/or D configuration, said mono- or polysaccharide having at least one hydroxyl function that must be free and/or optionally one or more optionally protected amine functions;

R is a linear or branched, saturated or unsaturated alkyl radical, or a cycloalkyl ring member having from 1 to 14 carbon atoms, or a phenyl radical, said radical, said ring member or said phenyl radical optionally being substituted with at least one radical selected from among —OR′₁, —NR′″₁R′₂, —COOR″₂ and —CONHR′″₂;

R′, R₁ and R₂, which may be identical or different, have the same definition as that given for R, and can also represent a hydrogen or a hydroxyl radical;

R′₂ and R′″₂, which may be identical or different, represent a hydrogen atom, or a radical selected from among a hydroxyl radical and a linear or branched, saturated or unsaturated alkyl radical having from 1 to 8 carbon atoms;

R′₁, R″₁, R″₂ and R′″₁, which may be identical or different, represent a hydrogen atom, or a radical selected from among a linear or branched, saturated or unsaturated alkyl radical having from 1 to 8 carbon atoms;

the definitions of the other substituents of general formula (I) remaining unchanged.

According to another embodiment, preference will be given to the compounds of general formula (I) such that:

S is a monosaccharide or a polysaccharide having up to 2 sugar units, in pyranose and/or furanose form, and of the L and/or D configuration, said mono- or polysaccharide having at least one hydroxyl function that must be free and/or optionally one or more optionally protected amine functions;

X is a group selected from: —CO—, —CH(NR₁R₂)— and —CHR′;

the definitions of the other substituents of general formula (I) remaining unchanged or having the preferred values given above.

Advantageously, the preferred monosaccharides are selected from among D-glucose, D-mannose, D-xylose, D-lyxose, L-arabinose, L-rhamnose, D-glucuronic acid, D-galacturonic acid, D-iduronic acid, N-acetyl-D-glucosamine and N-acetyl-D-galactosamine, and more advantageously D-glucose, D-xylose, N-acetyl-D-glucosamine or L-fucose, and very preferably D-xylose.

Also advantageously, the preferred polysaccharides having up to 6 sugar units are selected from among D-maltose, D-cellobiose, D-maltotriose, a disaccharide combining a uronic acid selected from D-iduronic acid and D-glucuronic acid, with a hexosamine selected from D-galactosamine, D-glucosamine, N-acetyl-D-galactosamine and N-acetyl-D-glucosamine, and an oligosaccharide containing at least one xylose advantageously selected from among xylobiose, methyl-β-xylobioside, xylotriose, xylotetraose, xylopentaose and xylohexaose, and preferably xylobiose, which is composed of two xylose molecules linked by a 1,4-linkage.

Among the C-glycoside derivatives of formula (I) according to the invention, the following are most particularly preferred:

Compound 1. 1-(C-β-D-xylopyranosyl)propan-2-one:

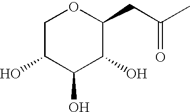

Compound 2. 1-(C-β-D-glucopyranosyl)propan-2-one:

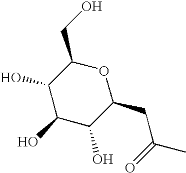

The administration of an agent for protecting and/or stimulating γδT lymphocytes of the skin and/or the mucous membranes results in increased release of IGF1 by the epidermal cells and promotes physiological proliferation of keratinocytes and/or decreases epidermal differentiation.

Given the fact that IGF1 release is dependent on GH and prolactin secretion and that these hormones are released in a pulsatile manner during the phase of falling asleep and only during the phase of falling asleep (it is a question of a waking state/sleep alternation and not a day/night alternation), the present invention also features the cosmetic use of at least one compound of general formula (I), for mimicking the effect of sleep on epidermal cell renewal and thus adding to the effect of sleep on the skin or supplementing the dysfunction of cutaneous epidermal functions which can appear when there is a lack of sleep.

The regime or regimen according to the invention will therefore more particularly be aimed at mimicking the effects of sleep on epidermal cell renewal in order to prevent and/or correct the characteristic cutaneous effects of a lack of sleep, and/or at stimulating the skin when there is a lack of sleep and inducing epidermal cell renewal.

Thus, administration according to the invention makes it possible to prevent and/or treat the cutaneous manifestations generated by the slowing down of cell renewal, thus results in cell regeneration in the epidermis and improves the appearance of the surface of the skin.

The consequences of this activity are that the administration of the compounds of general formula (I) according to the invention to an individual suffering from lack of sleep makes it possible more particularly to treat drawn and/or sunken features, in particular around the eyes, and to make the complexion uniform.

According to a second embodiment, the present invention features a cosmetic treatment process for effacing the cutaneous signs of fatigue by stimulating epidermal cell regeneration, characterized in that it comprises the application, to the face, of at least one compound of general formula (I).

The administration of at least one compound of general formula (I) also makes it possible to prevent and/or treat hair disorders such as a modification of hair density, quantity or quality, a consequence, for example, of a slowing down or an arrest of growth or a loss of hair follicles.

Thus, according to another embodiment, the present invention features the administration of at least one compound of general formula (I), for preventing thinning of the keratin fiber and/or inducing growth of the hair and/or of body hairs; for inducing the regrowth of hair or of body hairs which are more dense.

Thus, the invention also features the administration of at least one compound of general formula (I), as an agent for inducing and/or stimulating the growth of keratin fibers, hair or body hairs, in particular human keratin fibers, hair or body hair, and/or preventing their loss and/or increasing their density.

The expression “increasing the density of keratin fibers, and in particular hair density” means increasing the number of keratin fibers, in particular the number of hairs, per cm² of skin such as the scalp.

Thus, another application of at least one compound of general formula (I) relates to hair treatment compositions (shampoo, lotion, masks, etc.) for limiting and/or preventing hair loss and thus treating alopecia of any nature whatsoever and/or promoting the growth of healthy hair.

The cosmetic application according to the invention of a compound of general formula (I) can be carried out using a cosmetic care and/or makeup composition for keratin fibers.

The invention is also applicable to keratin fibers of mammals of the animal species (dog, horse or cat, for example).

The human keratin fibers to which the invention applies are in particular the hair, the eyebrows, the eyelashes, beard hair and moustache hair. More especially, the invention applies to human hair and/or eyelashes.

The present invention also features a cosmetic treatment process for human keratin fibers and/or for the skin from where said fibers emerge, including the scalp, useful in particular to stimulate the growth of human keratin fibers such as the hair and the eyelashes of a human being and/or prevent their loss, characterized in that it comprises applying, to the human keratin fibers and/or the skin from where said fibers emerge, a cosmetic composition comprising an effective amount of at least one compound of general formula (I), in leaving said composition in contact with the keratin fibers and/or the skin from where said fibers emerge, and optionally in rinsing the keratin fibers and/or said skin.

This treatment process has the characteristics of a cosmetic process insofar as it makes it possible to improve the aesthetics of the keratin fibers, and in particular of the hair and the eyelashes, by giving them greater vigor and an improved appearance. In addition, it can be used daily for several months, without medical prescription.

More especially, the present invention features a cosmetic care process for human hair and/or the human scalp, for the purpose of improving their condition and/or their appearance, characterized in that it comprises applying, to the hair and/or the scalp, a cosmetic composition comprising at least one compound of general formula (I), in leaving said composition in contact with the hair and/or the scalp, and optionally in rinsing the hair and/or the scalp.

This invention also features a cosmetic care and/or makeup process for human eyelashes, for the purpose of improving their condition and/or their appearance, characterized in that it comprises applying a mascara composition comprising at least one compound of general formula (I) and in leaving said composition in contact with the eyelashes. This mascara composition can be applied alone or as an undercoat for a conventional pigmented mascara and can be removed like a conventional pigmented mascara.

The compositions according to the invention can be administered orally, enterally or else topically; topical administration will be preferred.

In the case of oral administration, the compositions can be in any suitable form, such as an oral solution, gelatin capsules, sugar-coated tablets, soft or hard capsules, tablets to be swallowed or chewed, granules to be dissolved, a syrup, a solid or liquid food, etc.

The composition may also be in the galenic forms conventionally used for topical application, and in particular in the form of dispersions of the lotion or serum type, emulsions with a liquid or semi-liquid consistency of the milk type, obtained by dispersion of a fatty phase in an aqueous phase (O/W) or conversely (W/O), or suspensions or emulsions with a soft, semi-solid or solid consistency, of the cream or gel type, or alternatively multiple (W/O/W or O/W/O) emulsions, microemulsions, nanoemulsions, vesicular dispersions of ionic and/or non-ionic type, or wax/aqueous phase dispersions. These compositions are prepared to the usual methods.

It may also be in the form of a transdermal system for an active or passive transdermal release of the active agent(s), for example of the patch or gel patch (hydrogel) type.

The compositions according to the invention can thus constitute a treatment or care composition for the skin (including the scalp), keratin fibers (hair, eyelashes, eyebrows), nails or lips, or an antisun or artificial tanning composition, alternatively a cleansing or makeup-removing product for the skin, the hair, the eyebrows or the eyelashes, a deodorant product or a fragrancing compound. It is then generally uncolored or faintly colored, and it may optionally contain cosmetic or dermatological active agents. It may then be used as a care base for the skin or the lips (lip balms for protecting the lips against the cold and/or the sun and/or the wind), or as a day or night cream for facial and/or body skin. It may also be in the form of a treating or non-treating, coloring or non-coloring shampoo, or a conditioner.

The compositions according to the invention may also constitute a colored cosmetic composition, and in particular a makeup composition for the skin, keratin fibers (hair or eyelashes) and/or the mucous membranes, in particular a foundation, a blusher, a face powder, an eyeshadow, a concealer stick, a lipstick or a lip gloss, optionally having care or treating properties. Preferably, it may be a colored (beige or green) makeup composition for use in correcting the color of the complexion.

Depending on the purpose of the composition according to the invention, it may also comprise cosmetic or dermatological active agents which will be selected by those skilled in the art such that they do not harm the effect of the compounds of general formula (I) according to the invention.

In the context according to the invention for preventing and/or treating hair disorders, the compositions will be for cosmetic use, and in particular for topical application to the skin and keratin fibers, and more especially to the scalp, the hair and the eyelashes; they may be in any of the known galenic forms suitable for the method of use, for example, those indicated above.

In particular, the composition for application to the scalp or the hair may be in the form of a hair care lotion, for example for daily or twice-weekly application, a shampoo or a hair conditioner, in particular for twice-weekly or weekly application, a liquid or solid scalp cleaning soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for application to the eyelashes or body hairs, the composition of the invention may be in the form of a pigmented or unpigmented mascara, to be applied to the eyelashes with a brush or alternatively to beard or moustache hair.

According to a specific embodiment, the composition according to the invention is in the form of a hair cream or lotion, a shampoo or hair conditioner, a hair mascara or an eyelash mascara.

According to a specific embodiment of the invention, at least one additional active compound for the hair, that promotes the regrowth and/or that limits the loss of keratin fibers, and in particular of the hair, can be combined with the compound of general formula (I).

Said active agent for the hair may be selected from among:
anti-seborrhoeic agents, such as certain sulfur-containing amino acids, 13-cis-retinoic acid, cyproterone acetate;
agents for combating squamous states of the scalp (dandruff), such as zinc pyrithione, selenium disulfide, climbazole, undecylenic acid, ketoconazole, piroctone olamine (octopirox) or ciclopiroctone (ciclopirox);
active agents that stimulate hair regrowth and/or promote slowing of hair loss, more particularly;
nicotinic acid esters, including in particular tocopheryl nicotinate, benzyl nicotinate and C₁-C₆ alkyl nicotinates such as methyl nicotinate or hexyl nicotinate;
pyrimidine derivatives, such as 2,4-diamino-6-piperidinopyrimidine 3-oxide or “Minoxidil” described in U.S. Pat. Nos. 4,139,619 and 4,596,812; aminexil or 2,4-diaminopyrimidine 3-oxide described in WO 96/09048;
agents that are both lipoxygenase inhibitors and cyclooxygenase inducers, or cyclooxygenase-inducing agents that promote hair regrowth, such as those described by the applicant in EP-0,648,488;
antibiotics such as macrolides, pyranosides and tetracyclines, and in particular erythromycin;
cinnarizine, nimodipine and nifedipine;
hormones, such as estriol or analogues, or thyroxine and salts thereof;
anti-androgenic agents, such as oxendolone, spironolactone, diethylstilbestrol and flutamide;
cromakalim and nicorandil.

The compositions according to the invention can be applied to the alopecic areas of the scalp and the hair of an individual, and optionally left in contact for several hours and optionally rinsed off.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Demonstration of the Induction of γδT Lymphocyte Proliferation by C-Glycoside Derivatives of General Formula (I):

The γδT lymphocyte proliferation-inducing activity is tested in the following way: human peripheral blood cells are cultured in the presence of an RPMI-type culture medium supplemented with L-glutamine (2 mM), penicillin/streptomycin (50 μg/50 IU/ml) and foetal calf serum (10%). The C-glycoside derivatives 1 and 2 are added at various concentrations (10 to 0.05 mM), along with phytohemagglutin (PHA at 5*G/ml), a positive control for total lymphocyte proliferation. After 3 days of culture, the proliferation is revealed by BrdU labeling.

The results obtained are the following:

| | % stimulation relative | | | | | |
|---|---|---|---|---|---|---|
| Active agent | to the control | | | | | |
| 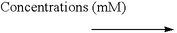 | 10 | 5 | 1 | 0.5 | 0.1 | 0.05 |
| Compound 1: 1-(C-β-D- | 74* | 106* | 131 | 84 | 115 | 108 |
| xylopyranosyl)propan-2-one | | | | | | |
| Compound 2: 1-(C-β-D- | 224 | 227 | 123 | 90 | 76 | 64 |
| glucopyranosyl)propan-2-one | | | | | | |

*cytotoxicity

All the derivatives tested exhibit a high human lymphocyte proliferation capacity starting from 1 mM.

### EXAMPLE 2

### Compositions According to the Invention:

Makeup-removal lotion for the face:

| | |
|---|---|
| Compound 2 | 1.00% |
| Strontium chloride | 5.00% |
| Antioxidant | 0.05% |
| Isopropanol | 40.00% |
| Preservative | 0.30% |
| Water | qs 100%. |

Facial care gel:

| | |
|---|---|
| Compound 1 | 5.00% |
| Vichy spring water | 10.00% |
| Thickening polymer | 1.00% |
| Antioxidant | 0.05% |
| Isopropanol | 40.00% |
| Preservative | 0.30% |
| Water | qs 100%. |

“Sleep” repair cream:

| | |
|---|---|
| Compound 1 | 1.00% |
| Glyceryl stearate | 2.00% |
| Polysorbate 60 (Tween 60 sold by the company ICI) | 1.00% |
| Stearic acid | 1.40% |
| Triethanolamine | 0.70% |
| Carbomer | 0.40% |
| Liquid fraction of shea butter | 12.00% |
| Perhydrosqualene | 12.00% |
| Antioxidant | 0.05% |
| Preservative | 0.30% |
| Water | qs 100%. |

Wax/water mascara:

| | |
|---|---|
| Beeswax | 6.00% |
| Paraffin wax | 13.00% |
| Hydrogenated jojoba oil | 2.00% |
| Water-soluble film-forming polymer | 3.00% |
| Triethanolamine stearate | 8.00% |
| Compound 2 | 1.00% |
| Black pigment | 5.00% |
| Preservative | qs |
| Water | qs 100%. |

This mascara is applied to the eyelashes like a conventional mascara, with a mascara brush.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for maintaining and/or reestablishing the balance from epidermal cell proliferation and differentiation, comprising administering to an individual in need of such treatment, a thus effective amount of at least one compound having the following general formula (I): 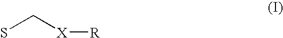
in which:
S is a monosaccharide or a polysaccharide containing up to 20 sugar units, in pyranose and/or furanose form and of L and/or D configuration, said mono- or polysaccharide having at least one hydroxyl function that is necessarily free and/or optionally one or more optionally protected amine functions;
the S—CH₂X bond is a bond of C-anomeric type;
X is a group selected from: —CO—, —CH(NR₁R₂)—, —CHR′—, —C(═CHR)—;
R is a linear or branched, saturated or unsaturated alkyl, perfluoroalkyl or hydrofluoroalkyl chain, or a cycloalkyl, cycloperfluoroalkyl or cyclohydrofluoroalkyl ring member having from 1 to 18 carbon atoms, or a phenyl radical, said chain, said ring member or said phenyl radical optionally being interrupted with one or more heteroatoms selected from among oxygen, sulfur, nitrogen and silicon, and optionally substituted with at least one radical selected from —OR′₁, —SR″₁, —NR′″₁R′₂, —COOR″₂, —CONHR′″₂, —CN, halogen, perfluoroalkyl and hydrofluoroalkyl and/or at least one optionally substituted cycloalkyl, aryl or heterocyclic radical;
R′, R₁ and R₂, which may be identical or different, have the same definition as that given for R, and can also represent a hydrogen or a hydroxyl radical;
R′₂ and R′″₂, which may be identical or different, represent a hydrogen atom, or a radical selected from among a hydroxyl radical and a linear or branched, saturated or unsaturated alkyl, perfluoroalkyl and/or hydroxyfluoroalkyl radical having from 1 to 20 carbon atoms;
R′₁, R″₁, R″₂ and R′″₁, which may be identical or different, represent a hydrogen atom, or a radical selected from among a linear or branched, saturated or unsaturated alkyl, perfluoroalkyl and/or hydroxyfluoroalkyl radical having from 1 to 20 carbon atoms;
with the following provisos:
R₁ and R₂ cannot simultaneously each be a hydroxyl radical;
R′″₁ and R′₂ cannot simultaneously each be a hydroxyl radical;
when S is a D-xylose and X is ═CO, then R cannot be an unsubstituted phenyl;
when S is a D-glucose and X is the function —OH, then R cannot be a p-hydroxyphenyl; and
when S is a D-glucose and X is ═O, then R cannot be a p-methoxyphenyl or a hexyl radical.

2. The regime or regimen as defined by claim 1, wherein general formula (I) S is a monosaccharide or a polysaccharide having up to 5 sugar units, in pyranose and/or furanose form, and of the L and/or D configuration, said mono- or polysaccharide having at least one hydroxyl function that is necessarily free and/or optionally one or more optionally protected amine functions.

3. The regime or regimen as defined by claim 1, wherein general formula (I) S is a polysaccharide having up to 6 sugar units selected from the group consisting of D-maltose, D-cellobiose, D-maltotriose, a disaccharide combining a uronic acid selected from among D-iduronic acid and D-glucuronic acid, with a hexosamine selected from among D-galactosamine, D-glucosamine, N-acetyl-D-galactosamine and N-acetyl-D-glucosamine, or an oligosaccharide selected from the group consisting of xylobiose, methyl-β-xylobioside, xylotriose, xylotetraose, xylopentaose and xylohexaose.

4. The regime or regimen as defined by claim 1, wherein general formula (I) S is a monosaccharide or a polysaccharide having up to 2 sugar units, in pyranose and/or furanose form, and of the L and/or D configuration, said mono- or polysaccharide having at least one hydroxyl function that is necessarily free and/or optionally one or more optionally protected amine functions.

5. The regime or regimen as defined by claim 1, wherein general formula (I) S is a monosaccharide selected from the group consisting of D-glucose, D-mannose, D-xylose, D-lyxose, L-arabinose, L-rhamnose, D-glucuronic acid, D-galacturonic acid, D-iduronic acid, N-acetyl-D-glucosamine, N-acetyl-D-galactosamine, and L-fucose.

6. The regime or regimen as defined by claim 1, wherein general formula (I) R is a linear or branched, saturated or unsaturated alkyl chain, or a cycloalkyl ring member having from 1 to 14 carbon atoms, or a phenyl radical, said chain, said ring member or said phenyl radical optionally being substituted with at least one radical selected from among —OR′₁, —NR′″₁R′₂, —COOR″₂ and —CONHR′″₂.

7. The regime or regimen as defined by claim 1, said at least one compound having the general formula (I) being selected from the group consisting of:
Compound 1. 1-(C-β-D-xylopyranosyl)propan-2-one; and
Compound 2. 1-(C-β-D-glucopyranosyl)propan-2-one.

8. The regime or regimen as defined by claim 1, for mimicking the effects of sleep on epidermal cell renewal.

9. The regime or regimen as defined by claim 1, for correcting the cutaneous effects caused by lack of sleep.

10. The regime or regimen as defined by claim 1, for inducing epidermal cell renewal.

11. The regime or regimen as defined by claim 1, for inducing epidermal cell regeneration.

12. The regime or regimen as defined by claim 1, for improving the appearance of the surface of the skin and/or treating drawn or sunken features and/or making the complexion uniform.

13. The regime or regimen as defined by claim 1, for preventing thinning of the keratin fibers and/or inducing growth of head hair and/or of body hairs.

14. The regime or regimen as defined by claim 1 , for inducing the regrowth of head hair or of body hairs which are more dense.

15. The regime or regimen as defined by claim 1, for preventing and/or treating head hair loss and/or the loss of body hairs.

16. The regime or regimen as defined by claim 1, for preventing and/or treating alopecia.

17. The regime or regimen as defined by claim 1, comprising co-administering at least one active agent selected from the group consisting of agents for preventing hair loss and/or agents for activating the regrowth of head hair and of body hairs.

18. The regime or regimen as defined by claim 1 for effacing the cutaneous signs of fatigue by stimulating epidermal cell regeneration, comprising topically applying onto the face of an individual in need of such treatment, a thus effective amount of said at least one compound of general formula (I).

19. A cosmetic treatment regime or regimen for human keratin fibers and/or for the skin from where said fibers emerge, including the scalp, to stimulate the growth of human keratin fibers and/or prevent their loss, comprising:
topically applying, to the human keratin fibers and/or the skin from where said fibers emerge, a cosmetic composition comprising at least one compound of general formula (I) as defined in claim 1,
leaving said composition in contact with the keratin fibers and/or the skin from where said fibers emerge, and
optionally, rinsing the keratin fibers and/or said skin.

20. A regime or regimen for protecting and/or stimulating the activity and/or the proliferation of gamma-delta T lymphocyte cells (γδT cells), and/or inducing the secretion and the release of IGF1 in the skin, comprising administering to an individual in need of such treatment, a thus effective amount of at least one compound having the following general formula (I): 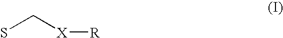
in which:
S is a monosaccharide or a polysaccharide containing up to 20 sugar units, in pyranose and/or furanose form and of L and/or D configuration, said mono- or polysaccharide having at least one hydroxyl function that is necessarily free and/or optionally one or more optionally protected amine functions;
the S—CH₂X bond is a bond of C-anomeric type;
X is a group selected from: —CO—, —CH(NR₁R₂)—, —CHR′—, —C(═CHR)—;
R is a linear or branched, saturated or unsaturated alkyl, perfluoroalkyl or hydrofluoroalkyl chain, or a cycloalkyl, cycloperfluoroalkyl or cyclohydrofluoroalkyl ring member having from 1 to 18 carbon atoms, or a phenyl radical, said chain, said ring member or said phenyl radical optionally being interrupted with one or more heteroatoms selected from among oxygen, sulfur, nitrogen and silicon, and optionally substituted with at least one radical selected from —OR′₁, —SR″₁, —NR′″₁R′₂, —COOR″₂, —CONHR′″₂, —CN, halogen, perfluoroalkyl and hydrofluoroalkyl and/or at least one optionally substituted cycloalkyl, aryl or heterocyclic radical;
R′, R₁ and R₂, which may be identical or different, have the same definition as that given for R, and can also represent a hydrogen or a hydroxyl radical;
R′₂ and R′″₂, which may be identical or different, represent a hydrogen atom, or a radical selected from among a hydroxyl radical and a linear or branched, saturated or unsaturated alkyl, perfluoroalkyl and/or hydroxyfluoroalkyl radical having from 1 to 20 carbon atoms;
R′₁, R′₁, R″₂ and R′″₁, which may be identical or different, represent a hydrogen atom, or a radical selected from among a linear or branched, saturated or unsaturated alkyl, perfluoroalkyl and/or hydroxyfluoroalkyl radical having from 1 to 20 carbon atoms;
with the following provisos:
R₁ and R₂ cannot simultaneously each be a hydroxyl radical;
R′″₁ and R′₂ cannot simultaneously each be a hydroxyl radical;
when S is a D-xylose and X is ═CO, then R cannot be an unsubstituted phenyl;
when S is a D-glucose and X is the function —OH, then R cannot be a p-hydroxyphenyl; and
when S is a D-glucose and X is ═O, then R cannot be a p-methoxyphenyl or a hexyl radical.
